(19) United States
(12) Patent Application Publication
(10) Pub. No.: US 2022/0118152 A1
(43) Pub. Date: Apr. 21, 2022

(54) USE OF LAYERED BLOOD PRODUCT FOR TREATING SURGICAL WOUNDS

(71) Applicant: REAPPLIX A/S, Birkerød (DK)

(72) Inventors: Rasmus LUNDQUIST, Brønshøj (DK); Niels Erik HOLM, Birkerød (DK); Morten Rosenberg ESKILDSEN, Allerod (DK)

(73) Assignee: REAPPLIX A/S, Birkerød (DK)

(21) Appl. No.: 17/507,269

(22) Filed: Oct. 21, 2021

Related U.S. Application Data
(60) Provisional application No. 63/094,694, filed on Oct. 21, 2020.

(51) Int. Cl. A61L 15/40; A61L 15/44; A61P 17/02; A61K 35/19; A61K 35/15

(52) U.S. Cl. CPC A61L 15/40; A61L 15/44; A61K 35/15; A61K 35/19; A61P 17/02

(57) ABSTRACT

A method for treating surgical wounds uses a blood product having a first layer, and a second layer, the first layer being adjacent to the second layer, the first layer defining a first outer surface of the blood product, the first layer having a majority of leukocytes and the second layer having a majority of thrombocytes. The method improves the healing or the wound and reduces the formation of scars.

### CROSS-REFERENCE TO RELATED APPLICATIONS

This application claims the benefit of U.S. Provisional Patent Application No. 63/094,694, filed Oct. 21, 2020, which is incorporated by reference herein in its entirety.

### BACKGROUND

### 1. Field of the Disclosure

The present disclosure relates to a method for treating surgical wounds in order to improve healing and reduce generation of scars.

### 2. Description of Related Art

The human coagulation system is able to stop bleeding and initiate healing. The function of the system to stop bleeding is well known and extensively investigated. However, the importance of coagulation products in the initiation of healing has been less investigated.

Blood products, such as fibrin sealants and platelets concentrate, are produced by isolating the platelet rich plasma (PRP) from anti-coagulated whole blood. The presence of platelets and plasma partly imitates the natural human coagulation system upon thrombin activation. This leads to a platelet containing autologous concentrate of growth promoting factors in a fibrin matrix. Such a composition can be used for covering wound surfaces and is claimed to initiate healing.

“Platelet-rich fibrin (PRF): A second-generation platelet concentrate. Part I: Technological concepts and evolution, Oral Surg Oral Med Oral Pathol Oral Radiol Endod 2006; 101:E37-44” by David M. Dohan et al describes how to prepare a platelet rich solid fibrin network from whole blood without adding any additives or reagents. The PRF protocol is: A blood sample is taken without anticoagulant in 10-mL glass tubes or glass coated plastic which are immediately centrifuged at approximately 400 g for 10 minutes. The absence of anticoagulant implies the activation in a few minutes of most platelets of the blood sample in contact with the glass tube walls and initiation of the coagulation cascades. Fibrinogen is initially concentrated in the plasma part of the tube before the circulating thrombin transforms it into fibrin. A fibrin clot is then obtained in the middle of the tube, extending from the upper part of the red corpuscles at the bottom of the tube to the acellular plasma at the top. Platelets are trapped massively in the fibrin meshes. The success of this technique entirely depends on the speed of blood collection and transfer to the centrifuge. Indeed, without anticoagulant, the blood samples start to coagulate almost immediately upon contact with the tube glass, and it takes a minimum of a few minutes of centrifugation to concentrate fibrinogen in the middle and upper part of the tube. Quick handling is the only way to obtain a clinically usable PRF clot. If the duration required to collect blood and launch centrifugation is overly long, failure will occur: The fibrin will polymerize before red cells has been separated and result in entire tube to be one blood clot. In conclusion, the PRF protocol makes it possible to collect a fibrin clot charged with serum and platelets. By removing the clot from the tube, manually cutting of the red cells part, and manually driving out the fluids trapped in the fibrin matrix (serum), practitioners will obtain autologous fibrin membranes.

However, this fibrin network includes some red thrombus containing a substantial part of red blood cells, which have to be manually cut off. In this step, part of the PRF will also be cut off. Furthermore, the components of the produced fibrin network, such as fibrin, leukocytes and thrombocytes, are arbitrary distributed and enmeshed within the product. The recovery of leukocytes is not described and at the low g force used, the recovery of leukocytes is low and as result, some will be located in the red cell part and will be lost. The enmeshment of cells within the fibrin leads to absent or slow release of these cells and thereby inhibits the contact-dependent anti-microbicidal potential of the included leucocytes.

“Oral Surg Oral Med Oral Pathol Oral Radiol Endod 2006; 101:E37-44 and 2006; 101:E45-50” by Dohan et al describes a network that does not represent a platelet concentrate in a shape and structure, which is directly applicable for covering wound surfaces. To obtain a shape and form/rigidity useable for covering wound surfaces and prevent red blood cell inclusion, the known platelet rich solid fibrin network will have to be reshaped manually and compressed. Furthermore, the method comprises several steps and cannot be prepared in one closed system and is therefore not convenient for clinical use.

“Cell separation in the buffy coat. Biorheology. 1988; 25(4):663-73” by Sutton et al describes how anti-coagulated full blood will separate into several layers upon centrifugation or passive sedimentation; Red blood cells, leukocytes and platelets (=buffycoat) and plasma. Further, by using centrifugation force of 10000 g for 10 minutes and using a float of density of 1.053, the buffy coat can be fixed by Glutaraldehyde, and removed for investigation; however, this cannot be used clinically due to the toxicity of the Glutaraldehyde. Several methods for extracting the buffycoat from anti-coagulated blood exists including the use of density defined substances (ie. Lymphoprep). In addition to the need for anti-coagulated blood the extracted cells will be suspended—and mixed (disorganized)—in the plasma that inevitably will be included.

EP 1637145A describes a method of filtration of cells from a suspension (e.g. blood cells including platelets and leukocytes) through a sheet like porous membrane, leaving the cells in the membrane as described. The sheet porous material can be prepared from fibrin. However, no layered structure is obtained, the cells are trapped in depth in the porous material and the use of allogeneic fibrin raises the risk of cross infection from other humans. Furthermore, the method comprises several steps and cannot be prepared in one closed system and is therefore not convenient for clinical use.

WO2010/020254, discloses a multilayered blood product comprising components from whole blood, especially fibrin, thrombocytes and leukocytes. The multilayered blood product is used for treatment of a wound where the blood product can improve healing of the wound.

Skin cancer is a disease that affects many peoples (e.g. in Denmark more than 15.000 patients are affected annually), and since it has been found that exposure to sun-light increases the prevalence of skin cancer the disease often affects the skin at areas exposed to sunlight, such as at the arms or in the facial area. Skin cancer is typically treated by surgery, where the affected cancerous skin is removed, and a wound is created. Since skin-cancer is often found on body parts that are visible in daily life, the healing of these wounds are a concern, in particular it is desired that these wounds do not give rise to scars after the healing or at least that scarring is reduced as much as possible.

Basal cell carcinoma (BCC) is the most common non-melanoma skin cancer. In Germany about 115,000 new cases are seen each year. The incidence has been calculated as 130 per 100,000 inhabitants and year.

About 80% of all BCC occur on the face, of these tumours 25% to 30% are found on the nose. BCC is the most common non-melanoma skin cancer of this region. The nose has a 2.5 times higher risk of recurrence of BCC after surgical excision. Localization on the nose is also considered a feature of high-risk BCC due its anatomical peculiarities and problems in pre-surgical identification of tumour margins. About one third of all incomplete BCC excisions are found on the nose. (Journal of Cutaneous and Aesthetic Surgery. 2014 July-September; 7(3) 143-150.

https://www.ncbi.nlm.nih.gov/pmc/articles/PMC4271293/)

Wounds after skin cancer surgery on the nose, such as on the nose wings, are a particular challenge, and experience shows that it is difficult to close the wound in an optimal way, leading to a high risk for delayed healing and infections, which not only is an inconvenience for the patient, but it also increases the risk for development of scar tissues leaving an undesirable visual impression after the healing is complete.

### SUMMARY OF THE DISCLOSURE

The object of the disclosure is to provide a new method for treating wounds, in particular surgical wounds, in order to reduce healing time and improve visual impression of the healed wound, giving rise to reduced scarring compared with other methods.

In a first aspect the present disclosure provides a method for surgical wound treatment comprising the steps of

a. Cleaning the wound from foreign material;

b. Removing fluids such as blood;

c. Applying a blood product to the wound, so the blood product covers the complete wound bed ensuring contact between the blood product and the intact skin boundary surrounding the wound; and

d. Covering the wound with a primary sheet;
wherein the blood product is selected among:

i. multilayered blood products comprising components from whole blood, especially thrombocytes and leukocytes, the blood product comprising a first layer, and a second layer, the first layer being adjacent to the second layer, the first layer defining a first outer surface of the blood product, the first layer comprising a majority of leukocytes and the second layer comprising a majority of thrombocytes; and wherein the blood product is oriented so the first layer is in direct contact with the wound; and

ii. single layered blood product consisting of components from plasma, com-ponents added to initiate a coagulation process and components created during the coagulation process.

According to the disclosure the surgical wound is after the surgical procedure cleaned for any foreign material and fluids like blood etc., are removed, and subsequently the wound is covered with the blood product so that a first layer, comprising a majority of leukocytes is contacting the wound; ensuring contact between the intact skin boundary adjacent to the wound and the blood product, and finally covering the blood product with a sheet in order to keep the blood product in contact with the wound surface.

The blood product is generated from a blood sample from a suitable donor, preferable from the patient itself.

Using the blood product for treating surgical wounds not only improves the healing of the wound but also reduces the formation of scars during the healing process, and therefore is the method particular suitable for use for treating wounds in body parts that are usually visible, such as wounds in the facial area, e.g. wounds on the nose or nose wings.

The disclosure is suited for treating surgical wounds, where the skin is completely or partially removed, and the tissues below are exposed. The disclosure is particular suited for treating wounds after skin cancer surgery.

When the blood product is applied to a wound to be treated it is important the blood product covers the complete wound bed ensuring contact between the blood product and the intact skin boundary surrounding the wound. Further, it is important that the first layer of the blood product comprising a majority of leukocytes are placed so it is in contact with the wound.

After the blood product has been placed on the wound is it covered with a sheet to secure that the blood product with the leukocytes and thrombocytes are kept in place and in contact with the wound surface and to protect the wound from the surroundings. The sheet can be made of fibrin or it can be a bandage.

In a preferred embodiment the sheet is made of fibrin and even more preferred, the blood product including the fibrin sheet is made as a multilayer product comprising a first layer comprising the majority of the leukocytes, a second layer comprising a majority of the thrombocytes and a third layer comprising a majority of the fibrin. Such a blood product is disclosed in WO 2010/020254A1 (incorporated herein by reference).

In a preferred embodiment the wound is further covered by a secondary protective bandage, preferably a nonadherence bandage as known in the art.

### The Blood Product

In a second aspect the present disclosure provides a blood product for use in a method for surgical wound treatment comprising the steps of

a. Cleaning the wound from foreign material;

b. Removing fluids such as blood;

c. Applying a blood product to the wound, so the blood product covers the complete wound bed ensuring contact between the blood product and the intact skin boundary surrounding the wound; and

d. Covering the wound with a primary sheet;

wherein the blood product is selected among:

i. multilayered blood products comprising components from whole blood, especially thrombocytes and leukocytes, the blood product comprising a first layer, and a second layer, the first layer being adjacent to the second layer, the first layer defining a first outer surface of the blood product, the first layer comprising a majority of leukocytes and the second layer comprising a majority of thrombocytes; and wherein the blood product is oriented so the first layer is in direct contact with the wound; and

ii. single layered blood products consisting of components from plasma, com-ponents added to initiate a coagulation process and components created during the coagulation process.

In one embodiment, the blood product comprises components from whole blood, especially thrombocytes and leukocytes, the blood product comprising a first layer and a second layer, the first layer being adjacent to the second layer and defining a first outer surface of the product and comprising a majority of leukocytes, the second layer comprising a majority of thrombocytes. Hereby, a blood product with a layered structure, comprising two or more layers, is provided, each layer provides different functionality due to each of the layer's different composition. The blood product is self-supporting, compact and solid, and the blood product has a structure, such that the blood product is directly applicable for the intended use. By majority is meant, that a component, such as thrombocytes or leukocytes, comprises at least 50%, 55%, 60%, 65%, 70%, 75%, 80%, 85%, 90%, 95%, 98%, 99% or even 100%, or any interval that can be defined from combinations of these mentioned percentages, volume- and/or mass wise of the respective layer and/or volume- and/or mass wise of the blood product. The first and the second layer are each continuous, but the thickness of the layers may vary and/or substantially parallel to each other, e.g. forming a stratified and/or multi-layered blood product. The blood product has preferably a maximum width/thickness-ratio of 1, 2, 3, 4, 5, or up to 10, 15, 20, 25, 30, 40, 50, 60, 70, 80, 90, 100 or even up to 500, where the width is measured along the layers of the blood product and the thickness is measured perpendicular to the layers of the blood product. In an embodiment the width/thickness ratio is in the range from 10-500, from 15-200 or from 20-200.

In one embodiment the blood product comprises mainly fibrin isolated from whole blood.

In another embodiment the blood product comprises mainly fibrin and platelets isolated from whole blood. The blood product is self-supporting, compact, and solid, and the blood product has a structure, such that the blood product is directly applicable for the intended use.

In another embodiment the blood product comprises mainly fibrin and platelets isolated from whole blood were the concentration of fibrin and platelets differs in the depth/thickness of the product such that the fibrin concentration is high on one side of the product and low on the other side. Likewise, the platelets concentration is high on the opposite side of the high fibrin concentration and low on the high fibrin concentration side.

In yet another embodiment the blood product comprises mainly fibrin, platelets and white cells isolated from whole blood were the concentration of fibrin, platelets and white cells differs in the depth/thickness of the product such that the fibrin concentration is high on one side of the product and low on the other side. Likewise, the platelets and white cells concentration is high on the opposite side of the high fibrin concentration and low on the high fibrin concentration side.

In a preferred embodiment, the blood product is the multilayered blood product disclosed in WO 2010/020254 A1 i.e., the blood product comprises components from whole blood, especially fibrin, thrombocytes and leukocytes, the blood product comprising a first layer, a second layer and a third layer, the second layer being adjacent to the first layer and the third layer, the first layer defining a first outer surface of the blood product and the third layer defining a second outer surface of the blood product, the first layer comprising a majority of leukocytes, the second layer comprising a majority of thrombocytes and the third layer comprising a majority of fibrin. Hereby, a blood product with a multilayered structure is provided, each layer provides different functionality due to each of the layer's different composition. The blood product is self-supporting, compact and solid, and the blood product has a structure, such that the blood product is directly applicable for the intended use. By majority is meant, that a component, such as fibrin, thrombocytes or leukocytes, comprises at least 50%, 55%, 60%, 65%, 70%, 75%, 80%, 85%, 90%, 95%, 98%, 99% or even 100%, or any interval that can be defined from combinations of these mentioned percentages, volume- and/or mass wise of the respective layer and/or volume- and/or mass wise of the blood product. The first, the second and the third layer are each continuous and/or substantially parallel to each other, e.g. forming a stratified and/or multilayered blood product. The blood product preferably consists of three layers, e.g. a first layer comprising a majority of leukocytes, a second layer comprising a majority of thrombocytes and a third layer comprising a majority of fibrin. The blood product has preferably a maximum width/thickness-ratio of 1, 2, 3, 4, 5, or up to 10, 15, 20, 25, 30, 40, 50, 60, 70, 80, 90, 100 or even up to 500, where the width is measured along the layers of the blood product and the thickness is measured perpendicular to the layers of the blood product.

In one embodiment, a majority of the leukocytes comprised in the blood product are comprised in the first layer. Hereby, a blood product is provided where the majority of the leukocytes comprised in the entire blood product is comprised in the first layer.

In another embodiment, a majority of the thrombocytes comprised in the blood product are comprised in the second layer. Hereby, a blood product is provided where the majority of the thrombocytes comprised in the entire blood product are comprised in the second layer.

If a third layer is present, a majority of the fibrin comprised in the blood product is comprised in the third layer. Hereby, a blood product is provided where the majority of the fibrin comprised in the entire blood product is comprised in the third layer.

In other embodiments, the blood product solely consists of components from whole blood. Hereby, a blood product is provided that solely consists of components from whole blood, meaning that no additives are added to the whole blood and/or the blood product, and that the blood product is directly derivable from whole blood, fractions of whole blood or combinations of fractions of whole blood.

In other embodiments, the blood product solely consists of components from plasma, added calcium chloride and components created during the coagulation process. Hereby, a single layered blood product is provided that solely consists of components from plasma, meaning that no additives except for calcium chloride are added to the product, and that the blood product is directly derivable from the plasma fractions of whole blood.

The blood product according to this embodiment may be prepared in advance of the surgical procedure and stored until use. For longer term storage it is preferred to dry the blood product immediately after preparation. The dry blood products can thereafter by unpacked and used during a subsequent surgical procedure.

Use of premade and stored blood products, e.g. dried blood products, has the clear advantage that an adequate supply can be prepared and put on stock before the surgical procedure in order to avoid the need for staff and equipment for preparation of the blood product during the surgical procedure.

Under all circumstances it is important to maintain sterility during the preparation and storage of any blood produce in order to avoid contamination and undesired growth during storage. Alternatively, the premade blood products may be sterilized using methods that do not denature fibrin, or only denature fibrin in a smaller extend, such as gamma radiation or ethylene oxide treatment.

Preferably, the blood product is autologous.

The blood product may be flexible. Hereby, a blood product is provided that can withstand applied stress during normal use without rupturing. Due to the flexibility of the blood product, the blood product conforms most continuous contours whereto the blood product is applied.

In another embodiment, the blood product further comprises a first substance chosen from a group comprising fibroblasts, keratinocyte cells and hyaluronic acid. Hereby, a blood product is provided which includes additional cells known to be important for skin regeneration and thereby further improves the healing potential of wounds, especially wounds in areas with low or unviable adjacent tissue. Hyaluronic acid, a known component of skin, has the potential to increase the water binding capacity of the blood product as well as increase the potential for incorporation/infiltration of the blood product in areas of tissue loss.

The disclosure also relates to, a blood product for therapeutic use and/or use of a blood product according to the aforementioned for therapeutic use.

The disclosure also relates to use of a blood product for manufacturing of a medicament for therapeutic use.

The disclosure also relates to a blood product for treatment of a wound and/or use of a blood product according to the aforementioned for treatment of a wound in order to reduce healing time and improve visual impression.

The disclosure also relates to use of a blood product for manufacturing of a medicament for treatment of a wound in order to reduce healing time and improve visual impression. Hereby, a blood product is provided which is particular suitable for manufacturing of a medicament for treatment of a surgical wound. By applying the first outer surface defined by the first layer against the wound, the wound is kept and/or maintained substantially sterile, e.g. free of infection, as the first layer comprises a majority of leukocytes, which are the first active cells and thus controls infection and attracts other cells including macrophages, while the second layer comprises a majority of thrombocytes which comprises growth promoting factors that stimulates the fibroblast cells.

If present, the third layer of the blood product comprises a majority of fibrin, and thus the second outer surface of the blood product provides an effective protection against contamination from the surroundings; the third layer furthermore comprises growth promoting factors, which is released over time. By applying the first outer surface defined by the first layer against the wound, the wound is kept and/or maintained substantially free of infection, as the first layer comprises a majority of leukocytes which are easily released from the product. Leukocytes are cells of the immune system defending the body against infection and foreign bodies, thus they control infection and further attracts other cells including macrophages. The second layer comprises a majority of thrombocytes which comprises growth promoting factors that stimulates the cells. As the leukocytes quickly will be released from the product, the second layer will face the wound surface for optimal delivery of growth promoting substances to the wound. The third layer of the blood product comprises a majority of fibrin, and thus the second outer surface of the blood product provides an effective protection against contamination from the surroundings; the third layer furthermore comprises growth promoting factors, which is released over time.

### Preparation of the Blood Product

The blood product may be prepared from a volume of whole blood, the method comprising the following steps: a) placing the volume of whole blood in a container means, the container means comprising a first material defining an inner surface in which the whole blood is in contact with, b) separating the whole blood into erythrocytes, serum and blood product by a centrifugal force acting on the whole blood placed in the container means, whereby the whole blood separates into layers comprising erythrocytes, blood product and serum due to the differences in densities between the erythrocytes, blood product and serum, the blood product comprising leukocytes and thrombocytes, the applied centrifugal force being at least 1000 times greater than the gravity force, e.g. g, acting on the whole blood, and d) removing the blood product from the container means.

In other embodiments the blood product is prepared from the plasma fraction of blood by a method comprising following steps: a) placing a volume of plasma in a container means; b) adding a component that initiate a coagulation process and letting the coagulation process proceed; c) applying a centrifugal force being at least 1000 times greater than the gravity force in a sufficient time to compact the coagulated material as a single layered product; and

d) removing the blood product from the container means. Alternatively, step b) is performed before step a).

Components that initiate a coagulation process are in general hydrophilic and/or polar surfaces such as kaolin, glass, iron etc. Further, it is important that Calcium ions are available for the process, so a typical way of initiating coagulation is adding calcium chloride and exposing the plasma to a hydrophilic and/or polar surface, e.g. an object composed of such a material or even the wall of the container means.

In a preferred embodiment the blood product is prepared according to the methods disclosed in WO 2010/020254, as outlined below.

The blood product may be prepared from a volume of whole blood, the method comprising the following steps: a) placing the volume of whole blood in a container means, the container means comprising a first material defining an inner surface in which the whole blood is in contact with, b) activating coagulation of the whole blood, c) separating the whole blood into erythrocytes, serum and blood product by a centrifugal force acting on the whole blood placed in the container means, whereby the whole blood separates into layers comprising erythrocytes, blood product and serum due to the differences in densities between the erythrocytes, blood product and serum, the blood product comprising fibrin, leukocytes and thrombocytes, the applied centrifugal force being at least 1000 times greater than the gravity force, e.g. g, acting on the whole blood, and d) removing the blood product from the container means. Hereby, a method is provided whereby a blood product is derivable from whole blood. The method can be processed in one cycle in a closed system, since there is no need for an isolation of the erythrocytes; however, the method can also be performed using an isolation of the erythrocytes. The yield of the method for extracting fibrin from the whole blood is at least above 10%, 20%, 30%, 40%, 50%, 60%, 70%, 80%, 90%, 95%, 97%, 98%, 99% or even 100%, while the yield of the method for extracting leukocytes from the whole blood is at least above 10%, 20%, 30%, 40%, 50%, 60%, 70%, 80%, 90%, 95%, 97%, 98%, 99% or even 100%, while the yield of the method for extracting thrombocytes from the whole blood is at least above 10%, 20%, 30%, 40%, 50%, 60%, 70%, 80%, 90%, 95%, 97%, 98%, 99% or even 100%. The obtainable blood product has a volume less than 30%, 20%, 15%, 10% or even less than 5% of the volume of the whole blood. Hereby, a method is provided whereby a blood product is obtainable by e.g. centrifugation giving rise to a centrifugal force. The applied centrifugal force is at least 1000, 2000, 3000, 4000, 5000, 6000, 7000, 8000, 9000, 10000, 11000, 12000, 13000, 14000, 15000, 16000, 17000, 18000, 19000 or even 20000 times greater than the gravity force, e.g. g, acting on the whole blood, or the applied centrifugal force is within any interval that can be defined from combinations of the mentioned numbers. The time the centrifugal force is applied for at least 30 seconds, 40 seconds, 50 seconds, 60 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes or 30 minutes, or the centrifugal force is applied within any interval that can be defined from combinations of the mentioned numbers.

The yield of the method for extracting fibrin from the whole blood is preferably at least 60%, or more.

The yield of the method for extracting leukocytes from the whole blood is preferably at least 50%, or more.

The yield of the method for extracting thrombocytes from the whole blood is preferably at least 60%, or more.

In one aspect of the method, the centrifugal force is applied for at least 30 seconds. The time the centrifugal force is applied for at least 30 seconds, 40 seconds, 50 seconds, 60 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes or 30 minutes, or the centrifugal force is applied within any interval that can be defined from combinations of the mentioned numbers.

In one embodiment, the blood product solely consists of components from whole blood.

Hereby, a method is provided whereby a blood product is derivable from whole blood, thus solely consisting of components from whole blood. Thus, the method is performed without adding any additives to the whole blood and/or the blood product.

In another aspect of the method, the coagulation in step b) is activated by the first material defining the inner surface. Hereby, a method is provided in which the coagulation is initiated when the whole blood is brought in contact with the inner surface, thus it can be avoided to add any object or the like to the whole blood to initiate coagulation.

In another aspect of the method, the coagulation in step b) is activated by exposing the whole blood to an object, such as a glass bead. Hereby, a method is provided in which the coagulation is initiated when the whole blood is brought in contact with the object added to the whole blood. Hereby, the coagulation can be initiated at a chosen point of time, which is optimal for the method.

In another aspect of the method, the first material of the inner surface of the container means is chosen from a group comprising of polypropylene, polyethylene, polycarbonate, polyimide, acrylonitrile butadiene styrene, styrene, modified styrene, polyurethane, polyester, polyethylene terephthalate and other polymer materials. The polymers in the mentioned group can furthermore be glass-filled. Hereby, a method is provided where a container means with an inner surface of a first material can be typical test tubes or the like made from all kinds of polymers, metal or glass. The material can also be chosen so the material property provides a minimal adhesive force/friction between the blood product and the inner surface. Polyamide and polyurethane are preferred as these materials initiates coagulation within a preferred level of activation which is higher than the level obtained using other polymers.

In another aspect of the method, the inner surface of the container means is surface treated, e.g. coated, in order to lower friction between the blood product and the inner surface of the first material. Hereby, a method is provided where a container means with an inner surface of a first material can be typical test tubes or the like made from all kinds of polymers, metal or glass. The inner surface can be surface treated and/or coated to obtain a minimal adhesive force/friction between the blood product and the inner surface.

In another aspect of the method, the centrifugal force is greater than an adhesive force acting between the inner surface and the blood product. Hereby, a method is provided where the centrifugal force is dominant as compared to the adhesive force, which secures a well-defined layered structure of the blood product. The centrifugal force can be at least 10, 100, 1000, 5000, 10000, 20000, 50000, 100000, 1000000 or even 10000000 times greater than the adhesive force, or the centrifugal force is within any interval that can be defined from combinations of the mentioned numbers. In another aspect of the disclosure, the centrifugal force and centrifugation time is of such strength that the adhesive force acting between the inner surface and the fibrin is broken/released and thereby allowing the fibrin layer to be compacted/compressed. Hereby, a method is provided where the centrifugal force is dominant as compared to the adhesive force, which secures a well-defined layered structure of the blood product. The centrifugal force needed to release the adhesion to the wall will depend on the fibrin density. The fibrin density will depend on several factors including coagulation activation, fibrin concentration, time, etc. The centrifugation force can be at least 10, 100, 1000, 5000, 10000, 20000, 50000, or even 100000 g, or the centrifugal force is within any interval that can be defined from combinations of the mentioned numbers.

In another aspect of the method, the blood product adhering to the inner surface is detached from the inner surface, at least once, during step c). Hereby, a method is provided where the possible adhesion of the blood product to the inner surface can be dealt with by separating the blood product at least once during step c). The separation can be done by mechanical means such as by cutting or the like. The compacting of the fibrin can then be performed at lower g-force as the g-force does not need to release the fibrin from the wall.

In another aspect, the method further comprises a compacting step, where the blood product is compacted by a compacting means, such as a filter placed in the container means. Hereby, a method is provided where the blood product can be compacted by e.g. a filter. The filter can be placed fixed or movable in the container means and the filter can be used to isolate the blood product.

In another aspect, the method further comprises an isolation step, where the erythrocytes are isolated from the blood product during step c). Hereby, a method is provided where the erythrocytes are isolated from the serum and the blood product during the method.

In another aspect, the method further comprises a washing step where the blood product is washed, so substantially all erythrocytes and/or serum attached to the blood product are detached. Hereby, a method is provided so the blood product is substantially clean from other components, that of the blood product itself. The serum formed during the centrifugation may be used as a washing fluid.

In another aspect of the method, the container means is a tube comprising an open end, closable by a detachable lid, and a closed end. Hereby, a method is provided where a stand- and test tube or the like can be used to perform the method.

In another aspect of the method, step b) precedes step a). Hereby, the coagulation can be activated before the whole blood is placed in the container means, thus the container means does not need to comprise any coagulation activator and/or the whole blood does not need to comprise a coagulation activator when the whole blood is placed in the container means. The coagulation can as an example, be activated during blood drawing by placing glass beads in the blood drawing tubing or choosing a tubing material that will activate blood. Thus, it can be avoided to add any object or the like to the whole blood to initiate coagulation.

In another aspect of the method, step b) occurs at least 1 minutes before step a). Hereby a method is provided where very fast handling is not necessary. Step b) can occur at least 30 seconds, 40 seconds, 50 seconds, 60 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes or even 20 minutes before step a). The level of activation of coagulation in step b) can also be controlled by the method. This allows the time between step b) and a) to be prolonged as the cell separation in step c) has to occur before fibrin levels is sufficient to inhibit cell separation.

In another aspect of the method, step b) occurs concurrently with step c). Hereby, the coagulation can be activated at an optimum point of time for the method during step c). The coagulation can be activated/initiated by a coagulation activator integrated into the container means, and/or by using a container material that will activate the blood. The coagulator material can be an object, such as glass beads, added to the whole blood in the container means.

In another aspect of the method, a first substance chosen from a group comprising fibro-blasts, keratinocyte cells and hyaluronic acid is added to the whole blood. Hereby, a blood product is obtainable which includes additional cells known to be important for skin regeneration and thereby further improves the healing of surgical wounds, especially wounds in areas with low or unviable adjacent tissue. Hyaluronic acid, a known component of skin, has the potential to increase the water binding capacity of the blood product as well as increase the potential for incorporation/infiltration of the blood product in areas of tissue loss.

In another aspect of the method, the compacting means, such as a filter, has a first fixed position and a second position. The filter is fixed in a position in the part of the container means containing the erythrocytes during the first part of the centrifugation where the leukocytes and thrombocytes has been separated, while the fibrin in the serum has not been compacted. Provided that the density of the filter is less than that of serum, a release of the filter will cause the filter to be transferred to the top of the tube and thereby collecting the leucocyte and thrombocyte layer and compacting the fibrin layer.

In another aspect of the method, the compacting means, such as a filter, is fixed in the first fixed position by a deformation in the container means wall. The filter is fixed by deforming the tube wall. The filter is released by removing the deformation of the wall.

In another aspect of the method, plasma comprising fibrin and a buffy coat comprising leukocytes and thrombocytes are used instead of whole blood. Hereby, a method is provided where whole blood excluding erythrocytes can be used, thus the blood product is directly derivable from whole blood, fractions of whole blood or combinations of fractions of whole blood.

In another aspect, the method is performed within 15 minutes. The method is performed within at least 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes 30 minutes; 40 minutes, 50 minutes, 60 minutes, 70 minutes, 80 minutes 90 minutes, 100 minutes or even within 120 minutes.

### BRIEF DESCRIPTION OF THE FIGURES

FIG. 1 shows a scientist displaying the single layer patch prepared in Example 1.

FIG. 2 shows the progress of healing of a nasal surgical wound using the blood product according to the disclosure. More details can be found in Example 2.

### EXAMPLES

### Example 1—Preparation of Single Layer Patch

A frozen plasma bag (450 ml) containing human plasma was thawed and calcium chloride was added to initiate coagulation.

After coagulation was completed, determined by visual inspection, the outlet tube was opened in one end and the whole plasma bag was compressed, by placing a flat surface with a heavy weight on the bag.

After compression was complete and the bag was flat, the bag was opened, and the single layer patch removed from the bag.

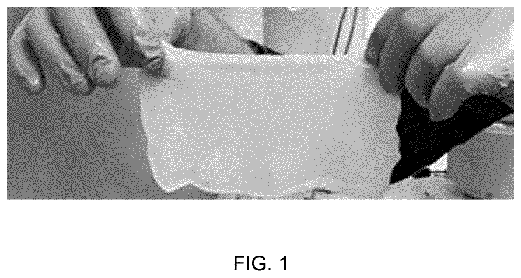

The generated single layer patch is illustrated on FIG. 1.

### Example 2—Nasal Skin Cancer Surgical Wound

A patient diagnosed with skin cancer on the nose wing participated in this example.

A blood sample was collected from the patient and a three layer blood product comprising a first layer comprising a majority of leukocytes, and a second layer comprising a majority of thrombocytes and a third layer comprising majority of fibrin was made by centrifugation according to the methods disclosed in WO 2010/020254A1. The blood product was collected as a patch and used for this example.

After the skin cancer was removed by surgery the wound was covered by the patch, securing that the patch was covering the whole wound bed and in contact with the intact skin surrounding the wound, and fastened by suture.

The healing of the wound was followed 3 months after the surgery, and it was seen that the wound healed well and after 3 months no scarring was observed.

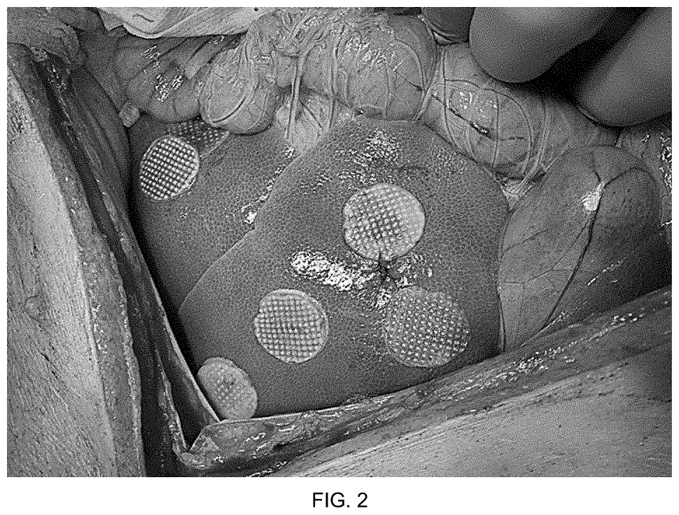

FIG. 2 shows pictures of the wound immediately after the surgery, after the patch has been sutures, after suture removal and after 3 months.

1. A method for surgical wound treatment comprising the steps of
a. cleaning the wound from foreign material;
b. removing fluids such as blood;
c. applying a blood product to the wound, so the blood product covers the complete wound bed ensuring contact between the blood product and an intact skin boundary surrounding the wound; and
d. covering the wound with a primary sheet;
wherein the blood product is selected among:
i. multilayered blood products comprising components from whole blood, especially thrombocytes and leukocytes, the blood product comprising a first layer, and a second layer, the first layer being adjacent to the second layer, the first layer defining a first outer surface of the blood product, the first layer comprising a majority of leukocytes and the second layer comprising a majority of thrombocytes; and wherein the blood product is oriented so the first layer is in direct contact with the wound; and
ii. single layered blood product consisting of components from plasma, components added to initiate a coagulation process and components created during the coagulation process.

2. The method according to claim 1, wherein the primary sheet covering the wound is fibrin or a bandage.

3. The method according to claim 1, wherein the blood product further comprises a third layer being adjacent to the second layer and defining a second outer surface of the blood product, wherein the third layer comprises a majority of fibrin.

4. The method according to claim 1, further comprising the step of:
e. covering the primary sheet with a secondary protective bandage.

5. The method of claim 4, wherein the secondary protective bandage is a non-adherence bandage.

6. The method according to claim 1, wherein steps a. to d. are repeated if healing is not initiated or complete after inspection days or weeks after first application.

7. The method according to claim 1, wherein the thrombocytes and/or the leukocytes are autologous.

8. The method according to claim 1, wherein the surgical wound is generated in a skin surgery procedure.

9. The method according to claim 8, wherein the skin surgery procedure is performed in a facial area.

10. The method according to claim 1, wherein the blood product is a single layered blood product consisting of components from plasma, components added to initiate a coagulation process and components created during the coagulation process.

11. The method according to claim 10, wherein the blood product is dried.

12. A blood product for use in a method for surgical wound treatment comprising the steps of
a. cleaning the wound from foreign material;
b. removing fluids such as blood;
c. applying a blood product to the wound, so the blood product covers the complete wound bed ensuring contact between the blood product and an intact skin boundary surrounding the wound; and
d. covering the wound with a primary sheet;
wherein the blood product is selected among:
i. multilayered blood products comprising components from whole blood, especially thrombocytes and leukocytes, the blood product comprising a first layer, and a second layer, the first layer being adjacent to the second layer, the first layer defining a first outer surface of the blood product, the first layer comprising a majority of leukocytes and the second layer comprising a majority of thrombocytes; and wherein the blood product is oriented so the first layer is in direct contact with the wound; and
ii. single layered blood products consisting of components from plasma, components added to initiate a coagulation process and components created during the coagulation process.

13. The blood product according to claim 12, further comprising a third layer being adjacent to the second layer and defining a second outer surface of the blood product, wherein the third layer comprises a majority of fibrin.

14. The blood product according to claim 12, wherein the thrombocytes and/or the leukocytes are autologous.

15. The blood product according to claim 12, wherein the surgical wound is generated in a skin surgery procedure.

16. The blood product according to claim 15, wherein the skin surgery procedure is performed in a facial area.

17. The blood product according to claim 12, wherein the blood product is a single layered blood product consisting of components from plasma, components added to initiate a coagulation process and components created during the coagulation process.

18. The blood product according to claim 17, wherein the blood product is dried.

19. The blood product according to claim 13, wherein the thrombocytes and/or the leukocytes are autologous.

20. The method according to claim 3, wherein the blood product is a single layered blood product consisting of components from plasma, components added to initiate a coagulation process and components created during the coagulation process.
